(19) United States
(12) Patent Application Publication
(10) Pub. No.: US 2025/0182861 A1
(43) Pub. Date: Jun. 5, 2025

(54) COMPUTATION SYSTEM AND COMPUTATION METHOD

(71) Applicant: NEC Corporation, Minato-ku, Tokyo (JP)

(72) Inventor: Takeshi AKAGAWA, Tokyo (JP)

(73) Assignee: NEC Corporation, Minato-ku, Tokyo (JP)

(21) Appl. No.: 18/841,430

(22) PCT Filed: Mar. 10, 2022
(86) PCT No.: PCT/JP2022/010564

§ 371 Date: Aug. 26, 2024

(51) Int. Cl. G16C 60/00; G09C 5/00

(52) U.S. Cl. CPC G16C 60/00; G09C 5/00

(57) ABSTRACT

To provide a computation system and a computation method for reducing the risk of inferring chemical compound data used for federated learning. A computation system includes: a concealment unit configured to perform a first processing of generating a model from a set of chemical compound data stored in each of a plurality of client terminals, and then concealing parameters of the generated model; and a secure computation unit configured to perform secure computation for integrating the generated models using the concealed parameters.

### TECHNICAL FIELD

The present disclosure relates to a computation system and a computation method.

### BACKGROUND ART

In recent years, in the field of pharmaceutical discovery and chemistry, it has been expected to link the data of structures of chemical compounds held by multiple organizations in order to reduce the development costs. Therefore, there is an expectation on the use of federated learning, in which machine learning is performed on the local side and the machine learning models are integrated on the server side.

Patent Literature 1 discloses a secure computation system in which calculation can be carried out with the data being concealed.

### CITATION LIST

### Patent Literature

Patent Literature 1: Japanese Patent No. 6795863

### SUMMARY OF INVENTION

### Technical Problem

By the way, it has been pointed out that a malicious user may obtain the parameters of a machine learning model and infer the chemical compound data which has been used for machine learning.

Thus, one of the objectives to be achieved in the example embodiments disclosed herein is to provide a computation system and a computational method that can reduce the risk of chemical compound data that has been used for federated learning being inferred.

### Solution to Problem

According to a first aspect of the present disclosure, a computation system includes:
concealment means for performing a first processing of generating a model from a set of chemical compound data stored in each of a plurality of client terminals, and then concealing parameters of the generated model; and
secure computation means for performing secure computation for integrating the generated models using the concealed parameters.

According to a second aspect of the present disclosure, a computation method includes:
performing a first processing of generating a model from a set of chemical compound data stored in each of a plurality of client terminals, and then concealing parameters of the generated model; and
performing secure computation for integrating the generated models using the concealed parameters.

### Advantageous Effects of Invention

According to the present disclosure, it is possible to provide a computation system and a computation method each adapted to reduce the risk of the chemical compound data that has been used for federated learning being inferred.

### BRIEF DESCRIPTION OF DRAWINGS

FIG. 1 is a block diagram showing a structure of an related computation system;

FIG. 2 is a block diagram showing an example of a configuration of a computation system according to a first example embodiment;

FIG. 3 is a block diagram showing an example of a functional configuration of a client terminal;

FIG. 4 is a block diagram illustrating an example of a functional configuration of a computation server;

FIG. 5 is a diagram illustrating an example of a computation method according to the first example embodiment;

FIG. 6 is a diagram illustrating an example of a computation method according to the first example embodiment;

FIG. 7 is a diagram illustrating an example of a computation method according to the first example embodiment;

FIG. 8 is a diagram illustrating an example of a computation method according to the first example embodiment;

FIG. 9 is a block diagram showing a functional configuration of a computation system according to the first example embodiment;

FIG. 10 is a block diagram showing an example of a computation system configuration according to a second example embodiment;

FIG. 11 is a block diagram illustrating an example of a functional configuration of a server;

FIG. 12 is a block diagram illustrating an example of a functional configuration of a computation server;

FIG. 13 is a block diagram illustrating an example of a functional configuration of a client terminal; and

FIG. 14 is a flowchart illustrating an example of an operation of a selection unit.

### EXAMPLE EMBODIMENT

### Background Leading to Example Embodiment

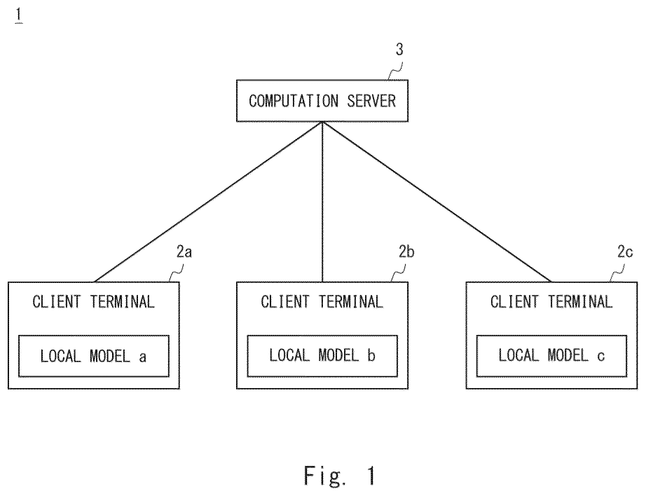

First, an overview of federated learning will be described. FIG. 1 is a block diagram showing a functional configuration of an related computation system 1. The computation system 1 includes client terminals 2a, 2b, and 2c, and a computation server 3.

The client terminal 2a generates a machine learning model (referred to as a local model a) from the data held by an organization A. The client terminal 2a transmits the parameters of the local model a to the computation server 3.

The client terminal 2b generates a machine learning model (referred to as a local model b) from the data held by an organization B. The client terminal 2b transmits the parameters of the local model b to the computation server 3.

The client terminal 2c generates a machine learning model (referred to as a local model c) from the data held by an organization C. The client terminal 2c transmits the parameters of the local model c to the computation server 3.

The computation server 3 generates a global model by integrating the local model a, the local model b, and the local model c. The computation server 3 may generate a global model by, for example, taking an arithmetic mean of the parameters. Note that the method of integrating the parameters is not limited to taking an arithmetic mean of the parameters. The computation server 3 transmits the global model to the client terminals 2a, 2b, and 2c.

With the computation system 1, the parameters of the local model a, the parameters of the local model b, and the parameters of the local model c are aggregated in one computation server 3, and there is a high risk of information leakage. The inventor of the present disclosure came up with the disclosure described in the first example embodiment based on the above study.

### First Example Embodiment

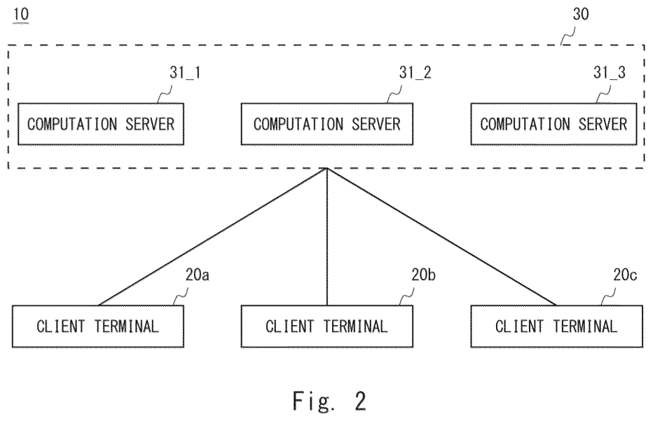

FIG. 2 is a schematic diagram showing an example of a configuration of a computation system 10 according to the first example embodiment. The computation system 10 includes client terminals 20a, 20b, and 20c and a computation server group 30. Each client terminal is a terminal of an organization (for example, pharmaceutical and chemical companies) using the computation system 1. The computation server group 30 includes computation servers 31_1, 31_2, and 31 3.

The client terminals 20a, 20b, and 20c are communicatively connected to the computation server group 30 via a network (not shown). The network may be wired or wireless. The network may be, for example, a VPN (Virtual Private Network).

In the following, when the client terminals 20a, 20b, and 20c are not distinguished from each other, they may simply be referred to as a client terminal(s) 20. Note that the number of the client terminals 2 is not limited to three, and may be two or may be equal to or greater than four. Similarly, when the computation servers 31_1, 31_2, and 31_3 are not distinguished from each other, they may simply be referred to as a computation server(s) 31. The number of the computation servers 31 is not limited to three, and may be two or may be equal to greater than four. In FIG. 2, the number of the client terminals 20 and the number of the computation servers 31 are the same, but they may not necessarily be the same.

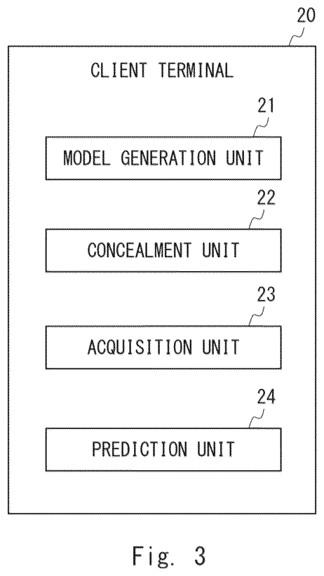

Next, description of the client terminal 20 will be given in detail with reference to FIG. 3. The client terminal 20 includes a model generation unit 21, a concealment unit 22, an acquisition unit 23, and a prediction unit 24.

The model generation unit 21 generates a local model from a set of chemical compound data held by an organization using the client terminal 20. The local model is also referred to as a local AI (Artificial Intelligence) model. The model generation unit 21 may use a set of chemical compound data as training data. The set of chemical compound data includes a plurality of items, for example, an item relating to the structure of a chemical compound and an item relating to the property of a chemical compound. A structure of a chemical compound is represented, for example, by a fixed-length bit string. Each bit in the bit string represents the presence or absence of a predetermined structure (for example, a benzene ring). A property of a chemical compound is expressed by a characteristic value (for example, the tensile strength value). A property value may be a value obtained through an experiment or a value obtained by simulation or theoretical computation. Since machine learning is performed at the client terminal 20, the chemical compound data held by an organization does not leak to any third party.

The set of chemical compound data typically includes items related to the purpose for which a chemical compound is utilized (headache medicine, abdominal pain medicine, etc.), items relating to the structure and the composition of the chemical compound, and items relating to theoretical computations and simulation results (for example, the simulation results of the property of a chemical compound). The set of chemical compound data further includes items relating to the process of preparing a chemical compound, items relating to materials informatics data (also referred to as machine learning data), and items relating to the function and the property of a chemical compound.

The concealment unit 22 divides each parameter of the local model into a plurality of shares and transmits the plurality of shares to the computation server group 30. Since the original parameter cannot be restored from one share, the client terminal 2 can be said to conceal the parameter.

The acquisition unit 23 acquires the global model from the computation result of the computation server group 30. The acquisition unit 23 acquires the global model by combining the computation results of the computation server 31_1, the computation server 31_2, and the computation server 31_3.

The prediction unit 24 uses the global model to predict the property and the structure of a chemical compound. The prediction unit 24 may, for example, use the global model to predict the property of a chemical compound from the structure of the chemical compound. The prediction unit 24 may use the global model to predict the structure of a chemical compound from the property of the chemical compound. The prediction unit 24 may output the prediction result to a display, monitor (not shown), or the like. The prediction unit 24 can predict the property of a chemical compound with high accuracy by using a global model.

The client terminal 20 includes a processor, a memory, and a storage device as a configuration not shown. The processor reads a computer program from the storage device into the memory and executes the computer program. Thus, the processor realizes the functions of the model generation unit 21, the concealment unit 22, the acquisition unit 23, and the prediction unit 24.

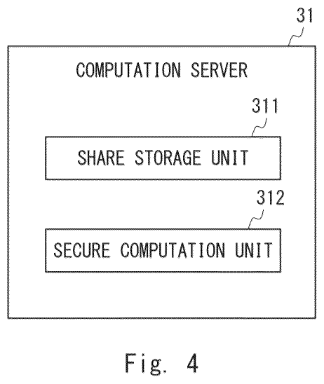

Next, the functions of the computation server 31 will be described in detail with reference to FIG. 4. The computation server 31 includes a share storage unit 311 and a secure computation unit 312.

The share storage unit 311 stores shares generated by the concealment unit 22 of the client terminal 20. Three shares generated for one parameter are separately stored in the share storage unit 311 of the computation server 31_1, the share storage unit 311 of the computation server 31_2, and the share storage unit 311 of the computation server 31_3.

The secure computation unit 312 performs secure computation for integrating the models using the shares stored in the share storage unit 311. The secure computation unit 312 may integrate the models at a predetermined time. The parameters of the local model are not known from the shares, so it can be said that the computation using the shares is a secure computation. The secure computation unit 312 of the computation server 31_1, the secure computation unit 312 of the computation server 31_2, and the secure computation unit 312 of the computation server 31_3 may cooperate to perform multiparty computation (MPC). The secure computation unit 312 transmits the computation result to the client terminal 20.

Like the client terminal 20, the computation server 31, too, includes a processor, a memory, and a storage device as a configuration not shown. The processor reads a computer program from the storage device into the memory and executes the computer program. Thus, the processor realizes the function of the secure computation unit 312.

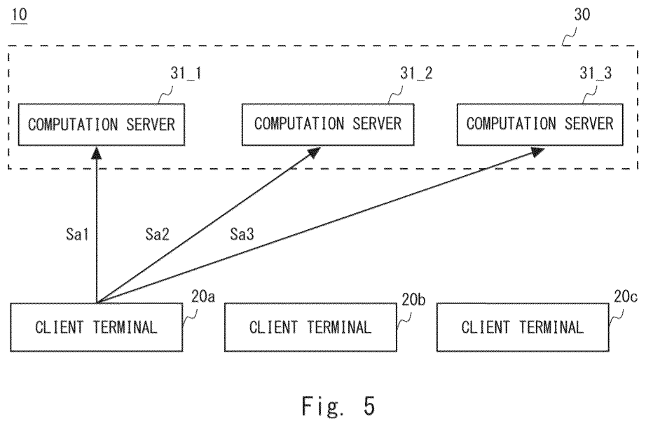

Next, the operation of the computation system 10 will be described concretely with reference to FIGS. 5 through 8. FIG. 5 is a diagram illustrating processing performed by the concealment unit 22 of the client terminal 20a. The concealment unit 22 of the client terminal 20a divides a parameter of the local model into shares Sa1, Sa2, and Sa3. The concealment unit 22 of the client terminal 20a transmits the share Sa1 to the computation server 31_1, the share Sa2 to the computation server 31_2, and the share Sa3 to the computation server 31_3.

Similarly, the client terminal 20b transmits a share Sb1 to the computation server 31_1, a share Sb2 to the computation server 31_2, and a share Sb3 to the computation server 31_3. Similarly, the client terminal 20c transmits a share Sc1 to the computation server 31_1, a share Sc2 to the computation server 31_2, and a share Sc3 to the computation server 31_3.

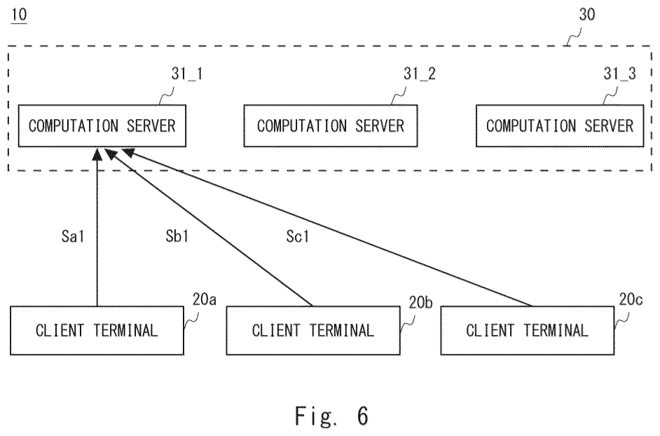

FIG. 6 is a diagram illustrating shares stored in the share storage unit 311 of the computation server 31_1. The share storage unit 311 of the computation server 31_1 stores the share Sa1 received from the client terminal 20a, the share Sb1 received from the client terminal 20b, and the share Sc1 received from the client terminal 20c.

The computation server 31_2 stores the share Sa2, the share Sb2, and the share Sc2 likewise. The computation server 31_3 stores the share Sa3, the share Sb3, and the share Sc3 likewise.

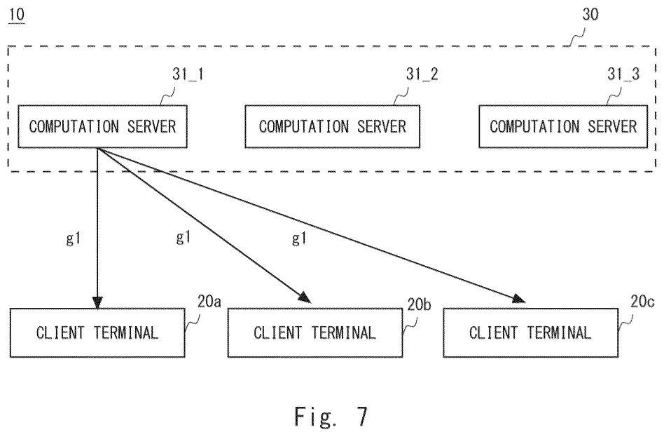

FIG. 7 is a diagram illustrating processing performed by the secure computation unit 312 of the computation server 31_1. The secure computation unit 312 of the computation server 31_1 performs computation for integrating the models using the shares Sa1, Sb1, and Sc1. The secure computation unit 312 of the computation server 31_1 transmits the computation result g1 to the client terminals 20a, 20b, and 20c.

The computation server 31_2 performs the same computation as that performed by computation server 31_1 using the shares Sa2, Sb2, and Sc2, and transmits the computation result g2 to the client terminals 20a, 20b, and 20c. The computation server 31_3 performs the same computation as that performed by the computation server 31_1 using the shares Sa3, Sb3, and Sc3, and transmits the computation result g3 to the client terminals 20a, 20b, and 20c.

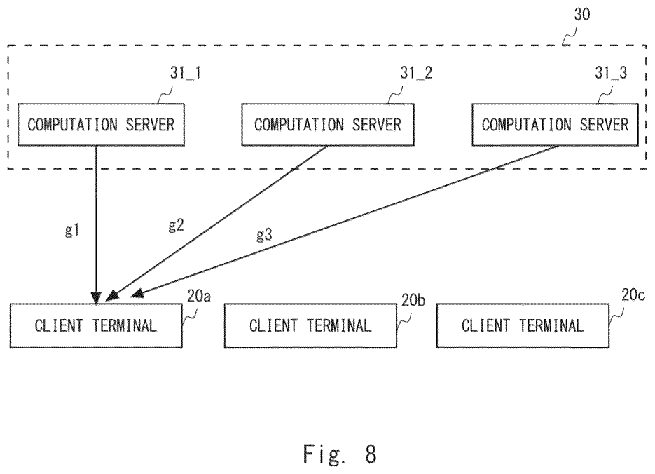

FIG. 8 is a diagram illustrating processing performed by the acquisition unit 23 of the client terminal 20a. The acquisition unit 23 of the client terminal 20a calculates the parameters of the global model from the computation result g1 of the computation server 31_1, the computation result g2 of the computation server 31_2, and the computation result g3 of the computation server 31_3. The acquisition unit 23 may, for example, calculate the sum of the computation results g1, g2, and g3. Similarly, the client terminals 20b and 20c can calculate the parameters of the global model. Note that any of the computation servers 31_1, 31_2, and 31_3 may calculate the parameters of the global model from the computation results g1, 2, and g3 and distribute them to the client terminals 20a, 20b, and 20c.

The computation system 1 can periodically update the global model by repeating the processings of FIGS. 5 through 8. The client terminal 20 first updates the global model and generates a new local model by performing machine learning using the new chemical compound data. Next, the client terminal 20 performs secret sharing of the parameters of the new local model. The client terminal 20 may perform secret sharing of the difference between the parameters of the local model and those of the global model. Next, the computation server group 30 executes secret computation.

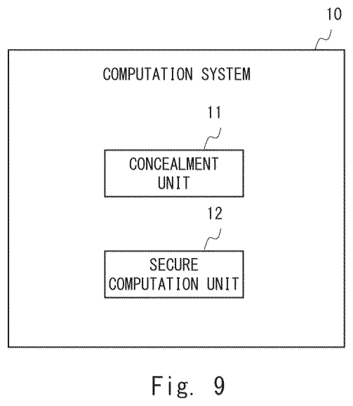

FIG. 9 is a block diagram showing the minimal functional configuration of the computation system 1. The computation system 1 includes a concealment unit 11 and a secure computation unit 12.

The concealment unit 11 performs a first processing of generating a model from a set of chemical compound data stored in each of a plurality of client terminals, and then concealing the parameters of the generated model. The concealment unit 22 of the client terminal 20 described above is a specific example of the concealment unit 11. When another server is provided in addition to the computation server group 30, the concealment unit 11 may be provided on another server. The concealment unit 11 may conceal the parameters of the local model by a method other than secret sharing (for example, homomorphic encryption).

The secure computation unit 12 performs secure computation for integrating the models using the concealed parameters. The secure computation unit 312 of the computation server 31_1, the secure computation unit 312 of the computation server 31_2, and the secure computation unit 312 of the computation server 31_3 function cooperatively as the secure computation unit 12. In addition, the secure computation unit 12 may perform secure computation on data encrypted by the homomorphic encryption method. In such a case, the computation system 1 may not have the computation server group 30.

Next, the effect of the computation system 1 will be described. In the computation system 1, federated learning is performed while keeping the parameters of the local model concealed. Thus, the risk of the chemical compound data used for learning in each organization being inferred from the parameters of the local model can be reduced.

In the secret computation, the computation can be executed using encrypted data, but there is a problem in that the execution time of the computation is long. However, the computational amount required for the integration of the local models is small enough that the computation system 1 can execute the secure computation in a realistic time.

The inventors and applicants have verified the accuracy and computation time of the computation system 1. The number of clients is set to two, secret sharing scheme is adopted for the secure computation method, and the number of distribution destination is set to three. It is verified that the computation system 1 can realize the same estimation accuracy in the same computation time as that of the related techniques.

### Second Example Embodiment

In the first example embodiment, the global model generated by the secure computation is distributed to each organization, and each organization uses the global model to predict the property of a chemical compound. Therefore, there is a risk that the organizations participating in federated learning will infer the chemical compound data used for learning from the global model. Therefore, it is preferable not to perform federated learning using highly confidential data.

In the first example embodiment, a processing of concealing the parameters of the local models (the first processing) is performed. However, there is a problem that the execution time of the secure computation is long, and it may be preferable to generate a global model without concealing the parameters of the local model. For example, in the case where the number of parameters of the local models are large, the execution time of the secure computation may be long. In addition, the execution time may be short in the case where the parameters are integrated by the arithmetic mean, but it may be long when the parameters are integrated by more complex computation. For example, when outliers of the parameters of the local model are considered, complex computation may be required.

The set of chemical compound data used for machine learning may include a plurality of items, as described above. The plurality of items may be, for example, an objective, structure, result of theoretical computation, preparation process, materials informatics, property, etc. These include items of a low confidentiality, such as results of theoretical computations, and items with a high confidentiality, such as objectives, structures, and preparation processes. These include items with a large data amount and a large number of model parameters, such as results of theoretical computations and data for materials informatics. In computation system according to the second example embodiment, processing to be performed on the data of each item is selected from a plurality of processings including the first processing.

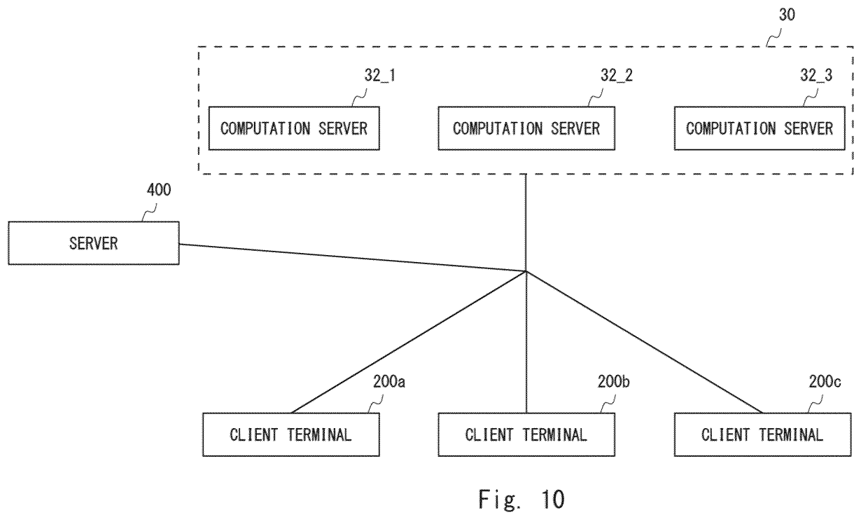

FIG. 10 is a block diagram showing the configuration of a computation system 100 according to the second example embodiment. The computation system 100 includes client terminals 200a, 200b, and 200c, the computation server group 30, and a server 400. Comparing the computation system 10 shown in FIG. 2 with the computation system 100, the server 400 is added to the computation system 100. The client terminals 20a, 20b, and 20c are replaced by the client terminals 200a, 200b, and 200c. The computation servers 31_1, 31_2, and 31_3 are replaced by computation servers 32_1, 32_2, and 32_3.

As in the first example embodiment, when the client terminals 200a, 200b, and 200c are not distinguished from each other, they may simply be referred to as a client terminal(s) 200. When the computation servers 32_1, 32_2, and 32_3 are not distinguished from each other, they may simply be referred to as a computation server(s) 32.

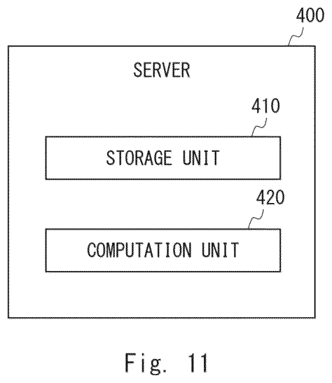

Next, the server 400 will be described in detail with reference to FIG. 11. The server 400 and the client terminal 200 are communicatively connected via a network (not shown).

The server 400 includes a storage unit 410 and a computation unit 420. The storage unit 410 stores data of each item received from the client terminal 200 (hereinafter also referred to as item-related data) and parameters of the local models.

The computation unit 420 has a function of performing computation using item-related data and a function of integrating parameters of the local models. The computation unit 420 executes computation in a state in which item-related data and parameters of the local models are not concealed.

First, a function for performing computation using the item-related data itself will be described. In the first example embodiment, the property of a chemical compound is predicted using a machine learning model, while the computation unit 420 predicts the property of a chemical compound using the item-related data. For example, in the case of predicting the property of a chemical compound having a certain structure, the property of the chemical compound can be predicted by calculating the average value of the property of chemical compounds having structures similar to the certain structure. As the computation using the item-related data is not limited to the computation of the average value, complex computation may be performed.

Next, the function of integrating the local models will be described. The computation unit 420 performs a processing which integrates the parameters stored in the storage unit 410 at a predetermined timing (e.g., once a day). Then, the computation unit 420 transmits the parameters of the global model to the client terminals 200a, 200b, and 200c.

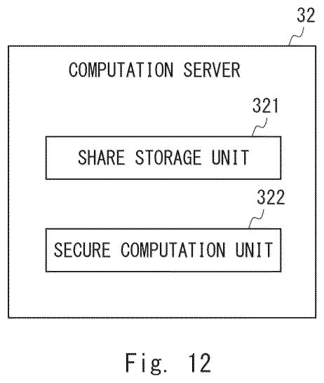

Next, the computation server 32 will be described with reference to FIG. 12. The computation server 32 includes a share storage unit 321 and a secure computation unit 322. Comparing the computation server 31 with the computation server 32 shown in FIG. 4, the share storage unit 311 is replaced by the share storage unit 321, and the secure computation unit 312 is replaced by the secure computation unit 322.

The share storage unit 321 stores the shares of item-related data in addition to the shares of the parameters of the local models. The share storage unit 321 may store the item-related data pertaining to a plurality of items. In such a case, not all the items need be concealed, but at least one item may be concealed.

In addition to the function of performing secure computation for performing integration of the local models, the secure computation unit 322 has the function of performing computation using shares of the item-related data stored in the share storage unit 321. The secure computation unit 322 executes secure computation in response to a computation request from the client terminal 200 and outputs the computation result. The secure computation unit 322 of the computation server 32_1, the secure computation unit 322 of the computation server 32_2, and the secure computation unit 322 of the computation server 32_3 may cooperatively perform multiparty computation.

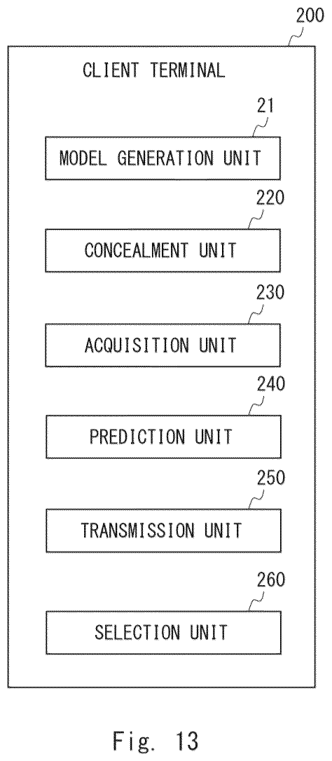

Next, the client terminal 200 will be described with reference to FIG. 13. Comparing the client terminal 20 with the client terminal 200 shown in FIG. 3, the concealment unit 22 is replaced by a concealment unit 220, the acquisition unit 23 is replaced by an acquisition unit 230, and the prediction unit 24 is replaced by a prediction unit 240. Further, a transmission unit 250 and a selection unit 260 are added.

The concealment unit 220 has a function of concealing item-related data in addition to a function of concealing parameters of a local model. The acquisition unit 230 has a function of acquiring a global model from the server 400 in addition to a function of acquiring a global model from the computation server group 30. The prediction unit 240 has a function of predicting the characteristics of a chemical compound using the global model and a function of predicting the characteristics of the chemical compound using the item-related data stored in the server 400 and the computation server group 30. The prediction unit 240 has a function of transmitting a computation request to the server 400 and the computation server group 30 and acquiring the computation result.

The transmission unit 250 has a function of transmitting the item-related data and the parameters of the local models to the server 400 without concealing the item-related data and the parameters of the local models.

The selection unit 260 selects a processing to be performed on the data of each item of the chemical compound data set from among the first processing, a second processing, a third processing, and a fourth processing. In the first processing, a local model based on the data of each item is generated, and then secret sharing of the parameters of the local model is performed. In the second processing, a local model based on the data of each item is generated, and then the parameters of the local model are transmitted to the server 400 without concealing the parameters. In the third processing, the data itself of each item is concealed. In the fourth processing, the data on the item is transmitted to the server 400 without concealing the data.

The selection unit 260 may select a processing to be performed on the data of each item of the set of chemical compound data from a plurality of processings including the first processing. The plurality of processings need not include all of the second processing, the third processing, and the fourth processing, but may include at least one of them.

When the first processing is performed, the model generation unit 21 generates a local model based on the item-related data, and the concealment unit 220 generates a plurality of shares based on the model parameters and transmits the generated plurality of shares to the computation server group 30. When the second processing is performed, the model generation unit 21 generates a local model based on the item-related data, and the transmission unit 250 transmits the model parameters to the server 400. When the third processing is performed, the concealment unit 220 generates a plurality of shares based on the item-related data and transmits the generated plurality of shares to the computation server group 30. When the fourth processing is performed, the transmission unit 250 transmits the item-related data to the server 400 without concealing the data relating to each item.

The selection unit 260 may select a processing to be performed according to the degree of the confidentiality of the data of each item. For example, the selection unit 260 may select the third processing or the fourth processing in which federated learning is not performed, instead of the first processing in which federated learning is performed, for an item of high confidentiality. . . . Further, the selection unit 260 may select the second processing, in which model parameters are concealed, instead of the first processing, in which model parameters are not concealed, for an item of low confidentiality.

The degree of confidentiality may be set for an item by the user operating the client terminal 200 at the time the chemical compound data is entered. In addition, the degree of confidentiality may be preset for each item of a set of chemical compound data.

In addition, the selection unit 260 may select whether the first processing, in which the parameter is concealed, or the second processing, in which the parameter is not concealed, is to be performed in accordance with the computational amount required for integrating the local models. When the computational amount required for integrating the local models is large (for example, when processing other than arithmetic operations is included therein, or when the number of parameters is large), the selection unit 260 may select the second processing instead of the first processing.

The computational amount required for integrating the local models may be determined according to the size of the item-related data. In addition, the computational amount required for integrating the models may be estimated in advance for each item.

In addition, the selection unit 260 may select which one of the third processing, in which the item-related data is concealed, or the fourth processing, in which the item-related data is not concealed, is to be performed according to the computational amount assumed for each item-related data. The selection unit 260 may select, of the third processing and the fourth processing, the fourth processing for items assumed to have large computational amount. The selection unit 260 may have a function of estimating the computational amount of the data of each item. The selection unit 260 determines a processing to be performed on the data of each item of the set of chemical compound data based on the result of the estimation.

The computational amount may be determined according to the computation contents assumed for each item. It is known that the secure computation can be carried out in a realistic time frame as long as the computation is mere arithmetic operations, but the logarithmic coefficient cannot be carried out in a realistic time frame. The selection unit 260 may select the fourth processing when the prediction unit 240 performs a computation request including processing other than arithmetic operations.

The selection unit 260 may select whether to perform the third processing or the fourth processing based on the time taken for the computation server group 30 to actually perform the computation. In such a case, the selection unit 260 transmits a portion of the data of each item to the computation server group 30, causes the computation server group 30 to actually perform predetermined computation (for example, calculating the average value), and measures the computational amount based on the result of the computation.

The selection unit 260 may select a processing to be performed on the data of each item while taking into account the desired processing time set for each item. For example, when the desired processing time is short, the selection unit 260 may select the fourth processing instead of the third processing. When the desired processing time is short, the first processing or the second processing may be selected. When the priority of confidentiality or computational amount is set for the data of each item, the selection unit 260 may determine the processing to be applied to the data of each item by taking the priority into account.

It may be determined in advance which processing is to be performed on data of each item of the set of chemical compound data. The selection unit 260 selects a processing to be performed on the data of each item of the set of chemical compound data based on the result of the determination.

The selection unit 260 may decide to perform the first processing to the data on the item relating to the property of the chemical compound. This is because the confidentiality of the data on the property of a chemical compound is not so high and the computational amount for integrating the local models is not likely to be large.

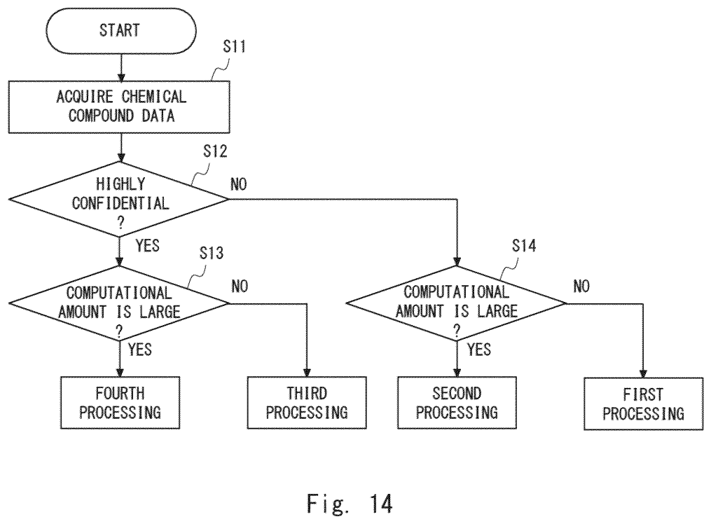

FIG. 14 is a flowchart illustrating an example of a method of selection performed by the selection unit 260. FIG. 14 is just an example. In FIG. 14, computational amount is determined after the degree of confidentiality of the data is determined, but the degree of confidentiality of data may be determined after computational amount is determined.

First, the selection unit 260 acquires a set of chemical compound data (Step S11). Next, the selection unit 260 determines whether or not each item-related data is highly confidential data (Step S12).

In the case where the data is highly confidential data (YES in Step S12), the selection unit 260 determines whether or not computational amount is large when the prediction unit 240 makes a prediction (Step S13). In the case where the computational amount is large (YES in Step S13), the selection unit 260 selects the fourth processing to transmit the item-related data to the server 400 without concealing the item-related data. In the case where the computational amount is not large (NO in Step S13), the selection unit 260 selects the third processing to conceal the item-related data and transmit the concealed item-related data to the computation server group 30.

In the case where the data is not highly confidential data (NO in Step S12), the selection unit 260 determines whether computational amount required for integrating the local models is large (Step S14). In the case where the computational amount is large (YES in Step S14), the selection unit 260 selects the second processing for transmitting the model parameters generated based on the item-related data to the server 400. In the case where the computational amount is not large (NO in Step S14), the selection unit 260 selects the first processing in which the model parameters generated based on the item-related data are concealed and output to the computation server group 30.

In the computation system 100 according to the second example embodiment, an optimal processing can be selected for processing each chemical compound data. According to the computation system 100, security can be improved because highly confidential data can be subjected to secret sharing and stored.

The program includes instructions (or software code) for causing the computer to perform one or more functions described in example embodiment when read into the computer. The program may be stored in a non-transitory computer-readable medium or a tangible storage medium. By way of example, and not a limitation, non-transitory computer readable media or tangible storage media can include a random-access memory (RAM), a read-only memory (ROM), a flash memory, a solid-state drive (SSD) or other types of memory technologies, a CD-ROM, a digital versatile disc (DVD), a Blu-ray (registered trademark) disc or other types of optical disc storage, and magnetic cassettes, magnetic tape, magnetic disk storage or other types of magnetic storage devices. The program may be transmitted on a transitory computer readable medium or a communication medium. By way of example, and not a limitation, transitory computer readable media or communication media can include electrical, optical, acoustical, or other forms of propagated signals.

The present disclosure has been described above with reference to the example embodiments, but the present disclosure is not limited by the foregoing. Various changes in the structure and details of the present disclosure can be made within the scope of the present disclosure that can be understood by a person skilled in the art.

Some or all of the above-mentioned example embodiment may be described as in the following supplementary notes, but not limited to the following.

### (Supplementary Note 1)

A computation system comprising:
concealment means for performing a first processing of generating a model from a set of chemical compound data stored in each of a plurality of client terminals, and then concealing parameters of the generated model; and
secure computation means for performing secure computation for integrating the generated models using the concealed parameters.

### (Supplementary Note 2)

The computation system described in Supplementary Note 1, further comprising selection means for selecting a processing to be performed on data of each item of the set of chemical compound data from a group of processings including the first processing and at least one another processing,
wherein the at least one another processing includes at least one of three processings of: a second processing of generating the local model based on the data of each item, and then transmitting the parameters to the server without concealing the parameters; a third processing of concealing the data itself of each item; and a fourth processing of transmitting the data of each item to the server without concealing the data.

### (Supplementary Note 3)

The computation system described in Supplementary Note 2, wherein the selection means is configured to select a processing to be performed on the data of each item according to the confidentiality of the data of each item and a computational amount assumed for the data of each item.

### (Supplementary Note 4)

The computation system described in Supplementary Note 3, wherein the selection means is configured to estimate the computational amount and select a processing to be performed on the data of each item based on the result of the estimation.

### (Supplementary Note 5)

The computation system described in Supplementary Note 4, wherein the selection means is configured to estimate the computational amount by actually performing a computation using a portion of the data of each item.

### (Supplementary Note 6)

The computation system described in Supplementary Note 3, wherein the selection means is configured to select a processing to be performed on the data of each item while taking into account a specified desired processing time.

### (Supplementary Note 7)

The computation system described in Supplementary Note 2, wherein
the set of chemical compound data includes an item relating to a structure of the chemical compound, an item relating to a result of simulation, an item relating to a process of preparing the chemical compound, and an item relating to a property of the chemical compound, and
which processing is to be performed on data of each item is predetermined.

### (Supplementary Note 8)

The computation system described in Supplementary Note 7, wherein the selecting means is configured to perform the first processing on an item relating to the property of the chemical compound.

### (Supplementary Note 9)

The computation system described in Supplementary Note 8, further comprising prediction means for predicting a property of a chemical compound from a structure of the chemical compound by using the model.

### (Supplementary Note 10)

The computation system described in Supplementary Note 8, further comprising prediction means for predicting a structure of a chemical compound from a property of the chemical compound by using the model.

### (Supplementary Note 11)

A computation method comprising:
performing a first processing of generating a model from a set of chemical compound data stored in each of a plurality of client terminals, and then concealing parameters of the generated model; and
performing secure computation for integrating the generated models using the concealed parameters.

### REFERENCE SIGNS LIST

1, 10,100 COMPUTATION SYSTEM
2, 2a, 2b, 2c, 20, 20a, 20b, 20c, 200, 200a, 200b, 200c CLIENT TERMINAL
a, b, c LOCAL MODEL
30 COMPUTATION SERVER GROUP
3, 31, 31_1, 31_2, 31_3, 32, 32_1, 32_2, 32_3 COMPUTATION SERVER
11, 22, 220 CONCEALMENT UNIT
311, 321 SHARE STORAGE UNIT
12, 312, 322 SECURE COMPUTATION UNIT
21 MODEL GENERATION UNIT
23, 230 ACQUISITION UNIT
24, 240 PREDICTION UNIT
250 TRANSMISSION UNIT
260 SELECTION UNIT
400 SERVER
410 STORAGE UNIT
420 COMPUTATION UNIT

What is claimed is:

1. A computation system comprising:
at least one memory storing instructions and
at least one processor configured to execute the instructions to:
perform a first processing of generating a model from a set of chemical compound data stored in each of a plurality of client terminals, and then concealing parameters of the generated model; and
perform secure computation for integrating the generated models using the concealed parameters.

2. The computation system according to claim 1, wherein the at least one processor is further configured to execute the instructions to:
select a processing to be performed on data of each item of the set of chemical compound data from a group of processings including the first processing and at least one another processing,
wherein the at least one another processing includes at least one of three processings of: a second processing of generating the local model based on the data of each item, and then transmitting the parameters to the server without concealing the parameters; a third processing of concealing the data of each item; and a fourth processing of transmitting the data of each item to the server without concealing the data.

3. The computation system according to claim 2, wherein the at least one processor is further configured to execute the instructions to:
select a processing to be performed on the data of each item according to the confidentiality of the data of each item and a computational amount assumed for the data of each item.

4. The computation system according to claim 3, wherein the at least one processor is further configured to execute the instructions to:
estimate the computational amount and select a processing to be performed on the data of each item based on the result of the estimation.

5. The computation system according to claim 4, wherein the at least one processor is further configured to execute the instructions to:
estimate the computational amount by actually performing a computation using a portion of the data of each item.

6. The computation system according to claim 3, wherein the at least one processor is further configured to execute the instructions to:
select a processing to be performed on the data of each item while taking into account a specified desired processing time.

7. The computation system according to claim 2, wherein
the set of chemical compound data includes an item relating to a structure of the chemical compound, an item relating to a result of simulation, an item relating to a process of preparing the chemical compound, and an item relating to a property of the chemical compound, and
which processing is to be performed on data of each item is predetermined.

8. The computation system according to claim 7, wherein the at least one processor is further configured to execute the instructions to:
perform the first processing on an item relating to the property of the chemical compound.

9. The computation system according to claim 8, wherein the at least one processor is further configured to execute the instructions to:
predict a property of a chemical compound from a structure of the chemical compound by using the model.

10. The computation system according to claim 8, wherein the at least one processor is further configured to execute the instructions to:
predict a structure of a chemical compound from a property of the chemical compound by using the model.

11. A computation method comprising:
performing a first processing of generating a model from a set of chemical compound data stored in each of a plurality of client terminals, and then concealing parameters of the generated model; and
performing secure computation for integrating the generated models using the concealed parameters.
